(19) United States
(12) Patent Application Publication
(10) Pub. No.: US 2020/0384285 A1
(43) Pub. Date: Dec. 10, 2020

(54) SELECTED LIGHT WAVELENGTHS FOR INCREASING SECRETION OF MEIBUM

(71) Applicants: Rolando Toyos, Germantown, TN (US); Melissa Toyos, Germantown, TN (US)

(72) Inventors: Rolando Toyos, Germantown, TN (US); Melissa Toyos, Germantown, TN (US)

(21) Appl. No.: 16/436,220

(22) Filed: Jun. 10, 2019

(51) Int. Cl. A61N 5/06; A61F 9/00

(52) U.S. Cl. CPC A61N 5/0613; A61N 5/0624; A61N 2005/0663; A61N 2005/0651; A61N 2005/0661; A61F 9/00

(57) ABSTRACT

Provided are processes for increasing secretion of meibum in a subject in need thereof, where the processes may include or be exclusively: applying light emitted from a treatment apparatus to an outer surface of one or more eyelids of the subject, the light emitted from the treatment apparatus having a predetermined set of photomodulating parameters, the light emitted from the treatment apparatus comprising or consisting of one or more wavelengths from 380 nm to 500 nm, and increasing secretion of meibum in the subject thereby. It was found that particular short wavelengths of light promote movement of meibum thereby treating or preventing one or more conditions of the eye related to suboptimal meibum secretion.

### TECHNICAL FIELD

This disclosure relates to increasing secretion of meibum in a subject in need thereof.

### BACKGROUND

Maintaining a proper balance of tear film components is essential for the health of the eye. Normal tear film requires proper amounts of lipid, aqueous and mucin components. The meibomian glands located in both the upper and lower eyelids include various sac-like acini that are responsible for secretion of lipid components of tear film known as meibum or sebum into the duct of the gland.

The meibum then passes into the orifices surrounded by smooth muscle tissue and the muscle of Riolan, which are presumed to aid in the expression of meibum onto the eye surface. It is believed that the meibomian gland orifices open during a normal blinking reflex to release meibum secretions onto the lid margin and then into the inferior tear meniscus. If the lipid secretions are balanced, an adequate lipid layer is maintained at the air interface to minimize evaporation and prevent dry eye or inflammatory states. However, if the lipid secretions are inadequate, the lipid layer also proves inadequate in minimizing evaporation leading to rapid evaporation. The resulting rapid evaporation may then lead to undesirable clinical consequences, such as blepharitis and/or dry eye syndrome.

Blockade of meibum secretions results in accelerated evaporation of the aqueous layer which, in turn, causes symptoms such as inflammation, dryness, scratching, irritation, burning, tearing, redness, and itchiness. These symptoms are typical of the pathological condition known as blepharitis, as well as dry eye syndrome. Uncorrected infection or dry eye syndrome may lead to aging effects, medication side effects, diseases, and eye structure problems.

Blepharitis is an eye condition characterized by chronic inflammation of the eyelid. The severity and time course of the condition can vary widely. Onset may be acute, resolving without treatment within two weeks to four weeks, but more commonly blepharitis results in long-lasting inflammation of the eyelid that may affect the eyelashes or tear production. Other symptoms may include dry eyes or red, itchy eyelids that may look greasy and crusted. Blepharitis can be classified as seborrhoeic, staphylococcal, mixed, posterior or meiobomitis, or parasitic.

Treatment of blepharitis typically includes eye drops, over the counter lid scrubs, or other cleansing agents. For treatment of posterior blepharitis, nutritional supplementation with omega 3-fatty acids or flaxseed oil may be prescribed. In some cases, antibiotic treatment may be also be recommended or required. However, these treatments may only be temporary, be cost prohibitive, or may require reapplication over a protracted time period.

Dry eye syndrome occurs when tears are not able to provide adequate moisture to the eye. Dry eye syndrome may result in the eye becoming dry, red, and inflamed. The main symptoms are discomfort and sensitivity to light. Treatment of dry eye syndrome typically includes eye drops, punctal occlusions, medications, and/or surgery. Most commonly, lubricating eye drops are used for treatment of dry eye syndrome. Several prescription medications are also available for increasing tear production. More recently, occlusions of the meibomian gland have been treated using heat and possibly gentle agitation to remove one or more occlusions of the meibomian gland and restore normal tear production. While these treatments may be somewhat effective at treating the condition, failure to adhere to a strict treatment regimen may result in chronic dry eye syndrome.

Thus, there is a need for new safe, effective, and affordable methods of treatment for increasing secretion of meibum to cure, treat, or prevent conditions such as meibomian gland dysfunction, blepharitis, or dry eye syndrome.

### SUMMARY

The following summary is presented to highlight particular aspects of the disclosure as described herein, and not as a limitation of the devices or processes of disclosure.

The processes and apparatuses of the present disclosure have utility as a treatment or prophylaxis for dry eye syndrome and increasing secretion of meibum from one or more meibomian glands of a subject in need thereof. Occlusions of the meibomian gland are treated applying light to the outer part of the eyelid at selected locations. As humans age, the areas of the skin tend to lose elasticity, suppleness and softness. Many of these effects may be attributed to a loss in skin collagens or a reduction in fibroblast activity.

Occlusions of the meibomian gland may be treated by applying light of particularly tailored wavelengths emitted from a treatment apparatus to an outer surface of one or more eyelids of the subject. The light may be applied to a lower eyelid or a portion thereof of the subject, optionally only the lower eyelid or a portion thereof of the subject, optionally excluding any region within the eye orbit. In some embodiments, the light is also applied to a periorbital region of the subject. In some embodiments, an upper eyelid may also be treated by exposure to light. Regardless of which area is targeted from treatment, the light applied to the subject has a predetermined set of photomodulating parameters. The inventors experimentally demonstrate that applying light having a predetermined set of photomodulating parameters the outer surface of one or more eyelids of the subject regenerates collagen and improves eyelid elasticity.

In embodiments, light is administered to the outer surface of one or more eyelids of the subject, wherein the light has a predetermined set of photomodulating parameters. The light, in some embodiments, is not applied to a meibomian gland via the inner eyelid or the region of the inner eyelid proximal to a meibomian gland. The light, in embodiments, is not applied to areas adjacent to an eyelid.

Administration of light to an outer surface of one or more eyelids may stimulate activity of the tear (i.e., lacrimal) glands situated in the upper and lower outer portions of each orbit/eyelid, thereby stimulating secretion of the aqueous layer of the tear film. The lacrimal gland produces tears, which may wet the surface of the eye reducing the dry eye.

An apparatus is provided that is specifically tailored to deliver light of a predetermined photomodulation to an outer surface of one or more eyelids, such as the lower eyelid, the upper eyelid, or both. The light source operates in a photomodulation mode to emit light of a predetermined photomodulation to activate fibroblasts to create collagen and elastin. The additional collagen and elastin functions to thicken and strengthen the eyelid region, thereby improving tone and stimulating movement of meibum onto the eye.

An apparatus may include one or more light sources, such as light emitting diodes (LEDs) that emit light with wavelengths being one of a group of wavelengths from 380 nm to 500 nm. Light emission may be provided to the subject either in a pulsed mode or a continuous mode for a treatment time. In embodiments where the light is pulsed, the pulses may be from 1 millisecond to 12 milliseconds with a delay between each pulse. The delay may be from 25 milliseconds to 75 milliseconds. The light may be pulsed from two times to five times.

In embodiments where the light is emitted in a continuous mode, the continuous operation for a treatment time may last from 1 minute to 4 minutes. The light source may emit light in a regularly or randomly alternating mode that optionally includes both a pulse mode and a continuous mode. The light emitted by an apparatus may have a fluence from 1 J/cm² to 10 J/cm². The light source may further include a structure that assists in the placement of the device such that the light source may be held directly on the surface of the subject's skin, or be held a selected distance from the skin.

The processes provided may be achieved using an apparatus as provided by the disclosure.

### DETAILED DESCRIPTION

The provided processes have utility as a treatment or prophylaxis of a condition resulting from suboptimal secretion of mebium. Processes are provided for increasing secretion of meibum from one or more meibomian glands, or by improving secretion of other eyelid glands in a subject in need thereof. A process, according to embodiments, includes applying light emitted from a treatment apparatus to an outer surface of one or more eyelids of the subject, the light emitted from the treatment apparatus having a predetermined set of photomodulating parameters comprising or consisting of one or more wavelengths, optionally from 380 nm to 500 nm, thereby increasing movement of meibum through a meibomian duct.

The inventor discovered that conditions caused by suboptimal transfer of meibum from a meibomian gland to the eye may be dramatically improved by modulating the reaction in an eyelid during a blink reflex. As humans age, the areas of the skin tend to lose elasticity, suppleness and softness. Many of these effects can be attributed to a loss in skin collagens or a reduction in fibroblast activity. By improving the condition of the skin of the eyelid, positive effects are transmitted to the underlying meibomian glands leading to improved meibum secretions and a reduction in symptoms of dry eye syndrome.

It is believed that symptoms relating to blepharitis, dry eye syndrome, or other conditions caused by sub-optimal transfer of meibum from a meibomian gland to the eye may be dramatically improved by modulating the reaction in an eyelid during a blink reflex. As humans age, certain areas of the skin tend to lose elasticity, suppleness, and softness. Many of these effects may be attributed to a loss in skin collagens or a reduction in fibroblast activity. By improving the condition of the skin of the eyelid, positive effects are transmitted to the underlying meibomian glands, leading to increased meibum secretion and a reduction in symptoms of a condition such as dry eye syndrome or blepharitis.

Skin and other body tissues have the ability to absorb light and use it as a source of energy to stimulate cellular activity. By carefully selecting the wavelength, fluence, duration, treatment times, and/or continuity of light applied to the outer surface of an eyelid, fibroblast activities may be significantly increased, which leads to greater production of collagen and elastin. These processes improve the structure of the eyelid by increasing meibum transfer through the meibomian duct and reducing the likelihood of blockage.

Without being limited by theory, it is believed that light of a particular wavelength, in the absence or presence of heat, may stimulate mitochondrial ATP production in fibroblasts in the skin. This ATP production then fuels increased collagen and elastin production, which regenerates skin elasticity. With increased collagen and elastin levels in the eyelid and optionally in the areas surrounding the eyelid, proper shaping of the skin is achieved during the blink reflex to promote normal meibum passage through the duct. Processes provided may be performed on subjects that do not have a current blockage of the meibomian gland duct and serve to proactively prevent such blockage.

In a process of preventing or treating blepharitis or dry eye syndrome in a subject, light is applied to the skin of an eyelid, and optionally the surrounding area, where the light is of a preselected wavelength. Illustrative wavelengths include those operable for increasing secretion of meibum in a subject.

The light emitted from the treatment apparatus may include one or more wavelengths from 380 nm to 500 nm, or any value or range therebetween. In embodiments, the light emitted from the treatment apparatus may include one or more wavelengths from 385 nm to 490 nm, from 390 nm to 480 nm, from 395 nm to 470 nm, from 400 nm to 460 nm, from 400 to 450 nm, from 405 nm to 440 nm, from 410 nm to 430 nm, from 410 nm to 420 nm, from 411 to 419 nm, from 412 nm to 418 nm, from 413 nm to 417 nm, or from 413 nm to 416 nm. In some embodiments, the light emitted from the treatment apparatus includes a wavelength of about 415 nm. The foregoing wavelengths are optionally +/−10 nm, optionally +/−5 nm. The light emitted from the treatment apparatus may increase the temperature of the skin and/or the meibomian glands. Optionally, the light emitted from the treatment apparatus does no increase the temperature of the skin and/or the meibomian glands, optionally does not increase the temperature by more than 2 degrees C. from the temperature prior to exposure to the light.

The light emitted from the treatment apparatus may have a fluence from 1 J/cm² to 20 J/cm² or any value or range therebetween. In embodiments, the light has a fluence from 2 J/cm² to 18 J/cm², from 3 J/cm² to 17 J/cm², from 4 J/cm² to 16 J/cm², from 5 J/cm² to 15 J/cm², from 6 J/cm² to 14 J/cm², from 7 J/cm² to 13 J/cm², from 8 J/cm² to 12 J/cm², or from 9 J/cm² to 11 J/cm². In certain embodiments, the light has a fluence of about 10 J/cm².

In embodiments, the light emitted from the treatment apparatus provides from 1 Joule of energy to 6 Joules of energy, or any value or range therebetween, to the one or more eyelids of the subject. In other embodiments, the light provides from 2 Joules of energy to 5 Joules of energy or from 3 Joules of energy to 4 Joules of energy to the one or more eyelids of the subject.

The light emitted from the treatment apparatus may be delivered to the one or more eyelids of the subject in either a pulsed or continuous emission format. In embodiments that utilize a pulsed light source, the light is optionally pulsed from 1 millisecond to 12 milliseconds, or any value or range therebetween. In other embodiments, the light is pulsed from 2 milliseconds to 10 milliseconds, from 3 milliseconds to 9 milliseconds, from 4 milliseconds to 8 milliseconds, or from 5 milliseconds to 7 milliseconds. In certain embodiments, the light is pulsed for about 6 milliseconds.

The light emitted from the treatment apparatus may also be pulsed multiple times per second resulting in a delay between each pulse. In embodiments, the light may be pulsed from two times to five times, from three times to five times, from four times to five times, from two times to four times, from three times to four times, or from two times to three times. In certain embodiments, the light is pulsed three times.

In embodiments that utilize a pulsed light source, the delay between each pulse is from 25 milliseconds to 75 milliseconds, or any value or range therebetween. In other embodiments, the delay between each pulse is from 30 milliseconds to 70 milliseconds, from 35 milliseconds to 65 milliseconds, or from 40 milliseconds to 60 milliseconds. In certain embodiments, the delay between each pulse is about 50 milliseconds.

Other embodiments may utilize a continuous light source. In embodiments that utilize the continuous light source, the application of the light emitted from the treatment apparatus may be applied to the subject from 1 minute to 8 minutes per treatment, or any value or range therebetween. In other embodiments, the application of the light emitted from the treatment may be applied to the subject from 1 minute to 7 minutes, from 1 minute to 6 minutes, from 1 minute to 5 minutes, from 1 minute to 4 minutes, or from 1 minute to 3 minutes. In certain embodiments, the application of the light emitted from the treatment lasts for about 2 minutes.

Light may be applied to the eyelid for a treatment time. A treatment time may be from one application time to a series of application times that are used daily, weekly, monthly, or as long as desired. Results are achieved after a single application time. Improved results are achieved by daily or weekly use of the light for one or more application times. The treatments may be performed for about four weeks or less if the treatments have shown an increase in secretion of meibum. Moreover, the treatments may be performed biweekly. In embodiments, the treatments are performed biweekly for four weeks, resulting in eight total treatments. It has been found that a continuous application time of three minutes at least twice a week produces excellent results in improving meibum secretion and treating blepharitis or dry eye syndrome.

In some embodiments corneal superficial punctate keratitis from the subject's baseline is reduced. Processes of reducing corneal superficial punctate keratitis include applying light to an outer surface of one or more eyelids or proximal thereto, the light having a predetermined set of photomodulating parameters, the light not directly applied to a meibomian gland or the inner surface of an eyelid, and reducing corneal superficial punctate by the applying. Photomodulating parameters as described herein also result in the treatment or prevention of blepharitis, result in the treatment or prevention of meibomian gland dysfunction, and/or result in the treatment or prevention of dry eye syndrome.

Further, the processes described herein may result in a reduction of the amount of bacteria on the outer surface of one or more eyelids of the subject. This reduction of the amount of bacteria may further result in the increased secretion of meibum through a meibomian duct. The reduction of the amount of bacteria on the outer surface of one or more eyelids of the subject may be especially effectuated if the one or more wavelengths of light include a wavelength or wavelengths from 380 nm to 420 nm, or other wavelength range or specific wavelength as provided herein.

The process as provided herein may further include applying a secondary light to the outer surface of one or more eyelids of the subject, the secondary light having a predetermined set of photomodulating parameters comprising emitting light, the secondary light comprising one or more wavelengths from 600 nm to 700 nm. Optionally, secondary light has a wavelength that is not in excess of 750 nm, optionally, not in excess of 700 nm, optionally not in excess of 690 nm. In some embodiments, secondary light has a wavelength of between 600 to 700 nm. Optionally, secondary light has a wavelength of 660 nm +/−10 nm. Optionally, secondary light has a wavelength that is 660 nm. Further non-limiting examples of secondary light are 605 nm, 630 nm, or 660 nm. Light source 116 could be populated by LEDs emitting a single wavelength, for example 660 nm, 630 nm or 605 nm and a combination of the above wavelengths. The bandwidth of the emitted light could be 20 nm or 40 nm as provided by a particular LED. The preselected wavelengths are far away from the infrared segment of the spectrum and do not cause increase in temperature of the treated skin. In embodiments, the secondary light may be applied simultaneously with the light applied from the treatment apparatus. The secondary light may be emitted from the same treatment apparatus or a separate treatment apparatus.

Regardless which of the process selected or treatment apparatus are used during any of the described processes, the treatment apparatuses may be held or positioned directly on the surface of the skin or may be held a selected distance from the skin. The distance of the skin from the treatment apparatus (i.e., the light emitting location) may be from 0.5 mm to 3 mm from the skin surface, from 1 mm to 3 mm from the skin surface, from 1 mm to 5 mm from the skin surface, or is directly in contact with the skin.

The light emitting location may be one or more light emitting diodes capable of emitting light of the desired predetermined set of photomodulating parameters. An exemplary apparatus design operable for achieving prevention or treatment by the processes of the disclosure and optionally delivering light of any one or all of the parameters herein is described in U.S. Pat. No. 10,272,256, although different photomodulating parameters are provided herein.

For example, an assembly of LEDs in a treatment apparatus may include different LEDs emitting in a relatively large spectrum from 380 nm to 2000 nm, although in some examples LEDs may be selected to emit light with preselected discrete wavelengths from 380 nm to 500 nm, or other wavelength range or specific wavelength as provided herein either directly or as is transmitted through a filter of chosen bandpass or cutoff wavelength. The one or more light sources may be populated by LEDs emitting a single wavelength, for example 400 nm, 415 nm, 430 nm, or a combination of the wavelengths. The bandwidth of the emitted light may be 5 nm, 10 nm, 20 nm or 40 nm as provided by a particular LED. The preselected wavelengths do not cause increase in temperature of the treated skin as the LEDs are optionally located far enough away from the skin to prevent such an increase in temperature.

The LEDs may emit light in a pulse mode or in a continuous emission mode. When LEDs operate to emit light in the pulse mode, they may emit pulses 1 millisecond to 12 milliseconds, or any value or range therebetween. The delay between each pulse may be from 25 milliseconds to 75 milliseconds, or any value or range therebetween. The pulses may be emitted in a predetermined pulse emitting pattern. The light may be pulsed from two times to five times. It should be understood that other pulsing patterns may be used without departing from the scope of the present method.

When LEDs operate to emit light in the continuous mode, the LEDs operate from 1 minute to 8 minutes. In both pulse and continuous operating mode the light emitted by the LEDs may have a fluence from 1 J/cm² to 20 J/cm².

The light may be applied to an eyelid or an area proximal to an eyelid. The light, in embodiments, is applied to a lower eyelid below the lash line without light being applied to an upper eyelid. The region proximal to an eyelid includes the orbital region of the face and may extend to the zygomatic region. In some aspects, the light is applied to the area of the lower eyelid below the lash line and excludes light application to the upper lid or areas proximal to the lower eyelid. The light, in embodiments, is not applied to or is prevented from application to the inner canthus, outer canthus, or both.

In some embodiments, the light is applied such that the temperature of the skin does not significantly increase. The skin temperature may be maintained during an application time within 2° C. of the skin temperature before treatment as presented upon rest at room temperature. In certain embodiments, the light is applied such that the temperature of the skin and/or meibomian glands is increased by at least 2° C. In some embodiments, heat is simultaneously applied with the light.

The processes of treating or preventing symptoms or causes of a conditions related to suboptimal meibum secretion as provided herein are appreciated not to apply light directly to the region of a meibomian gland such as the inner eyelid region. It was surprisingly discovered that light application as described herein may increase secretion of meibum without the need for direct stimulation of the gland itself as is typically done by massage, heat, or direct application of light (for heating purposes). The disclosure represents a much simpler process for treating or preventing dry eye syndrome or preventing meibomian gland blockade than was appreciated or envisioned in the art. As such, many embodiments of the disclosure exclude one or more of physical pulsation, application of heat, massage, sonic pulsation, or other prior methods of treating meibomian gland blockade or treatment of dry eye.

The inventors experimentally determined that light applied to at least one eyelid area, for example, to lower eyelid area and optionally the periorbital region of an eye activates fibroblasts to create collagen and elastin thereby thickening and strengthening the periorbital and eyelid area. The light may be applied to the lower eyelid area below the lash line without light being applied to the upper eyelid area. The region proximal to the lower eyelid area and/or the upper eyelid area optionally includes the orbital region of the face, which may extend to the zygomatic region. The light, in embodiments, is not administered or is prevented from contacting the skin in other than that of the lower eyelid area. New collagen production, elastin production, and combinations thereof improve eyelid tone (elasticity). The improvement in the eye lid tone (elasticity) facilitates eye blinking process and distribution of meibum and other fluids, such as tears, over the eye surface. Meibum (e.g., containing lipid) secretions form and maintain an adequate lipid layer at the air interface to minimize evaporation of aqueous content of the tears and prevent dry eye states.

The inventors have also identified that glands evacuate better from the upper eyelids following exposure of the upper eyelid skin to light. Without being bound to one particular theory, it is believed that this is related to gravity that assists evacuation of the tears and removed particles blocking the relevant duct(s). Accordingly, in some embodiments, an apparatus administers light to the upper eyelid area that may be from 20% to 30% less intensive then the treatment applied to the lower eyelid area, but sufficient to create a better platform for the upper eyelid particles blocking the meibomian duct evacuation during blinking.

Generally, the light source may be operated in an operation mode known as photomodulation. Photomodulation is a term that describes the use of low intensity light therapy to alter (enhance or reduce) the activity of living cells. It is a painless, non-invasive treatment that uses LEDs (or other light emitting device) to activate new collagen formation and simultaneously inhibit the breakdown of existing collagen, helping to rejuvenate the skin. New collagen formation, elastin production, and combinations thereof improve eyelid tone (elasticity). The improvement in the eyelid tone (elasticity) facilitates eye blinking process and distribution of meibum and other fluids, for example, tears over the eye surface. Meibum (lipid) secretions form and maintain an adequate lipid layer at the air interface to minimize aqueous tear component evaporation and prevent dry eye states.

Usually, light-based skin therapies, including intense pulsed light and laser treatments, rely on heat and thermal injury to create changes in skin appearance. Photomodulation does not use heat and there is no trauma to the tissues which means there is no downtime. However, there are specific sequences of light pulses that activate certain sub-cellular systems better than others. The reaction of the treatment recipients to photomodulation is individual. The photomodulating parameters may include pulse duration, time between the pulses, total light application time, and others.

According to one example, the light source may be held or located at a selected distance from skin. The distance may be between 1 mm and 5 mm. According to another example, the light source may instead be held directly on surface of skin and be in contact with the skin surface. Regardless of which distance is chosen, operation of the light source optionally does not change the temperature of the eyelids more than 2° C.

Optionally, the light source is an incandescent or halogen lamp covered by a filter that transmits a desired or selected wavelength (for example 400 nm, 415 nm, or 430 nm, or other wavelength or wavelength range as provided herein) and reflects or absorbs the other wavelengths and in particular wavelength that deliver heat that may increase the temperature of the eyelids. The filter may be a simple colored glass or a transparent glass with a dielectric coating. The filter may be transparent or having one of its sides processed to disperse light.

The processes of treating or preventing symptoms or causes of a condition related to suboptimal meibum secretion as provided herein are appreciated not to apply light directly to the region of a meibomian gland such as the inner eyelid region. It was surprisingly discovered that light application as described herein may increase secretion of meibum without the need for direct stimulation of the gland itself as is typically done by massage, heat, or direct application of light (for heating purposes). The disclosure represents a much simpler process for treating or preventing dry eye syndrome or preventing meibomian gland blockade than was appreciated or envisioned in the art. As such, many embodiments of the disclosure exclude one or more of physical pulsation, application of heat, massage, sonic pulsation, or other prior methods of treating meibomian gland blockade or treatment of dry eye.

The processes provide a simple, rapid, and effective means of treating or preventing blepharitis or dry eye syndrome.

Various aspects of the present disclosure are illustrated by the following non-limiting examples. The examples are for illustrative purposes and are not a limitation on any practice of the present disclosure. It will be understood that variations and modifications can be made without departing from the spirit and scope of the disclosure.

### EXAMPLES

### Example 1

Ten subjects were treated with a process that included applying light emitted from a treatment apparatus as described above to an outer surface of the lower eyelid. The light emitted from the treatment apparatus had a predetermined set of photomodulating parameters. The light emitted from the treatment apparatus had wavelengths ranging from 413 nm to 416 nm in a continuous emission format. Each subject was subjected to three treatments spanning two minutes each, for a total of six minutes. The apparatus did not administer heat or pressure to the eyelid, or apply light to either of the eye canthes. The results of the treatments are shown in Table 1.

TABLE 1
Reduction in Colony Forming Bacteria Units on Lower Eyelid.

| | Colony Forming | Colony Forming | Percent Reduction |
|---|---|---|---|
| | Bacteria Units | Bacteria Units | in Colony Forming |
| Subject | Before Treatment | After Treatment | Bacteria Units |
| 1 | 30 | 14 | 53% |
| 2 | 423 | 54 | 87% |
| 3 | 112 | 68 | 39% |
| 4 | 2 | 1 | 50% |
| 5 | 6 | 5 | 17% |
| 6 | 8 | 1 | 88% |
| 7 | 112 | 36 | 68% |
| 8 | 224 | 40 | 82% |
| 9 | 223 | 132 | 41% |
| 10 | 25 | 17 | 32% |
| Average | 116.5 | 36.8 | 68% |

The presence of colony forming bacteria units on a subject's eyelid are integrally associated with conditions like blepharitis, meibomian gland dysfunction, dry eye syndrome, or combinations thereof. A decrease in the number of colony forming bacteria units correlates with an increase in secretion of meibum in a subject, leading to treatment or prevention of the aforementioned conditions. As can be seen in Table 1, the average number of colony forming bacteria units on a subject's lower eyelid reduced by 68% after undergoing treatment. Therefore, it is believed that subjects treated with the processes described herein will show relief of one or more symptoms of dry eye syndrome, such as blepharitis, after just a single treatment and continue to improve over additional treatments.

### REFERENCE LIST

Almeida-Lopes L., Rigau J., Zangaro R. A., Guidugli-Neto J., Jaeger M. M. Comparison of the low level laser therapy effects on cultured human gingival fibroblasts proliferation using different irradiance and same fluence. Lasers Surg Med. 2001; 29(2):179-84.

Anderson R. R., Parrish J. A. The optics of human skin. J Invest Dermatol. 1981 July; 77(1):13-9.

Barolet, D., et al., Presented at the ASLMS annual meeting, Orlando, USA, 2005.

Bjerring P., Clement M., Heickendorff L., Lybecker H., Kiernan M. Dermal collagen production following irradiation by dye laser and broadband light source. J Cosmet Laser Ther. 2002 June; 4(2):39-43.

Goto E., Monden Y., Takano Y., Mori A., Shimmura S., Shimazaki J., Tsubota K. Treatment of non-inflamed obstructive meibomian gland dysfunction by an infrared warm compression device. Br J Ophthalmol. 2002; 86(12):1403-07.

Heickendorff L., Zachariae H., Bjerring P., Halkier-Sorensen L., Sondergaard K. The use of serologic markers for collagen synthesis and degradation in systemic sclerosis. J Am Acad Dermatol. 1995 April; 32(4):584-8.

Karu, T. Molecular mechanism of the therapeutic effect of low-intensity laser irradiation. Lasers in Life Science 1988; 2, 53-74.

Karu T. Primary and secondary mechanisms of action of visible to near-IR radiation on cells. J Photochem Photobiol B. 1999 March; 49(1):1-17. Review.

Kiistala U. Suction blisters device for separation of viable epidermis from dermis. J Invest Dermatol 1968: 50: 129-137.

Nussbaum E. L, Biemann I., Mustard B. Comparison of ultrasound/ultraviolet-C and laser for treatment of pressure ulcers in patients with spinal cord injury. Physical Therapy 1994(74):812-23.

Saperia, D., Glassberg, E., Lyons, R. F., Abergel, R. P., Baneux, P., Castel, J. C., Dwyer, R. M., and Uitto, J. (1986). Demonstration of elevated Type I and Type II procollagen mRNA levels in cutaneous wounds treated with heliumneon laser. Proposed mechanism for enhanced wound healing. Biochemical and Biophysical Research Communications 138, 1123-1128.

Skinner S. M., Gage J. P., Wilce P. A., Shaw R. M. A preliminary study of the effects of laser radiation on collagen metabolism in cell culture. Aust Dent J. 1996 June; 41(3):188-92.

Smith K. C. Photobiology and photomedicine: the future is bright. J Invest Dermatol. 1981 July; 77(1):2-7.

Sommer A. P., Pinheiro A. L., Mester A. R., Franke R. P., Whelan H. T. Biostimulatory windows in low-intensity laser activation: lasers, scanners, and NASA's light-emitting diode array system. J Clin Laser Med Surg. 2001, February; 19(1):29-33.

Ti-sapphire Laser, Wikipedia, en.wikipedia.org/wiki/Ti-sapphire_laser, accessed May 23, 2019.

Yu W., Naim J. O., Lanzafame R. J. The effect of laser irradiation on the release of bFGF from 3T3 fibroblasts. Photochem Photobiol. 1994 February; 59(2):167-70.

Webb C., Dyson M., Lewis W. H. Stimulatory effect of 660 nm low level laser energy on hypertrophic scar-derived fibroblasts: possible mechanisms for increase in cell counts. Lasers Surg Med 1998; 22(5):294-301.

Wheeland R. G. Lasers for the stimulation or inhibition of wound healing. Journal of Dermatologic Surgery & Oncology 1993(19):747-52.

Whelan H. T., Smits R. L. Jr, Buchman E. V., Whelan N. T., Turner S. G., Margolis D. A., Cevenini V., Stinson H., Ignatius R., Martin T., Cwiklinski J., Philippi A. F., Graf W. R., Hodgson B., Gould L., Kane M., Chen G., Caviness J., Effect of NASA light-emitting diode irradiation on wound healing., J Clin Laser Med Surg. 2001 December; 19(6):305-14. Review.

Zhang Y., Song S., Fong C. C., Tsang C. H., Yang Z., Yang M. cDNA microarray analysis of gene expression profiles in human fibroblast cells irradiated with red light. J Invest Dermatol. 2003 May; 120(5):849-57.

Various modifications of the present disclosure, in addition to those shown and described herein, will be apparent to those skilled in the art of the above description. Such modifications are also intended to fall within the scope of the appended claims.

Patents and publications mentioned in the specification are indicative of the levels of those skilled in the art to which the disclosure pertains. These patents and publications are incorporated herein by reference to the same extent as if each individual patent, application or publication was specifically and individually incorporated herein by reference.

The foregoing description of particular aspect(s) is merely exemplary in nature and is in no way intended to limit the scope of the disclosure, its application, or uses, which may, of course, vary. The embodiments are described with relation to the non-limiting definitions and terminology included herein. These definitions and terminology are not designed to function as a limitation on the scope or practice of the embodiments, but are presented for illustrative and descriptive purposes only. While the processes and devices are described as an order of individual steps or using specific arrangements of elements, it is appreciated that described steps or elements may be interchangeable such that the description of the embodiments include multiple parts or steps arranged in many ways as is readily appreciated by one of skill in the art.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

The foregoing description is illustrative of particular aspects of the disclosure, but is not meant to be a limitation upon the practice thereof.

What is claimed is:

1. A process for increasing secretion of meibum in a subject in need thereof, consisting essentially of:
applying light emitted from a treatment apparatus to an outer surface of one or more eyelids of the subject, the light emitted from the treatment apparatus having a predetermined set of photomodulating parameters, the light emitted from the treatment apparatus consisting of one or more wavelengths from 380 nm+/−10 nm to 500 nm+/−10 nm, and
increasing secretion of meibum in the subject to restore normal meibum passage through a duct thereby;
wherein the one or more eyelids of said subject are not heated above 2 degrees Celsius from physiological temperature.

2. The process of claim 1, wherein the light consists of one or more wavelengths from 400 nm+/−10 nm to 450 nm+/−10 nm.

3. The process of claim 1, wherein the applying is from 1 minute to 8 minutes per treatment.

4. The process of claim 3, wherein treatments are performed for four weeks.

5. The process of claim 3, wherein treatments are performed twice a week.

6. (canceled)

7. The process of claim 1, wherein the light comprises a fluence from 1 J/cm² to 20 J/cm².

8. The process of claim 1, wherein the light provides from 1 Joule of energy to 6 Joules of energy to the one or more eyelids of the subject.

9. The process of claim 1, wherein the light is pulsed.

10. The process of claim 9, wherein the light is pulsed for 1 millisecond to 12 milliseconds.

11. The process of claim 10, wherein there is a delay between each pulse.

12. The process of claim 11, wherein the delay is from 25 milliseconds to 75 milliseconds.

13. The process of claim 9, wherein the light is pulsed from two times to five times per second.

14. The process of claim 1, wherein the light is pulsed three times for 4 milliseconds to 8 milliseconds each with a delay of 40 milliseconds to 60 milliseconds between each pulse.

15. The process of claim 1, further comprising reducing corneal superficial punctate keratitis of the subject.

16. The process of claim 1, further comprising reducing the amount of bacteria on the outer surface of one or more eyelids of the subject.

17. The process of claim 1, further comprising treating blepharitis, meibomian gland dysfunction, dry eye syndrome, or combinations thereof.

18. The process of claim 1, wherein the light is supplied to the outer surface of one or more eyelids of the subject with a light-emitting diode.

19-20. (canceled)

21. The process of claim 1, wherein the light consists of one or more wavelengths from 413 nm+/−5 nm to 416 nm+/−10 nm.
